SEQUENCE LISTING (2 sequences)

SEQ ID NO: 1 - protein, 22 aa, synthetic construct
  source 1..22: note = amino acid sequence of modified cone snail polypeptide
NAICCGFPRSCASRNCRPHRCC

SEQ ID NO: 2 - protein, 22 aa, synthetic construct
  MOD_RES 1: note = Acetylation
NAICCGFPRSCASRNCRPHRCC

(19) United States
(12) Patent Application Publication
(10) Pub. No.: US 2025/0241837 A1
(43) Pub. Date: Jul. 31, 2025

(54) MODIFIED CONE SNAIL POLYPEPTIDE, PREPARATION METHOD AND USE THEREOF

(71) Applicant: SOUTHERN MARINE SCIENCE & ENGINEERING GUANGDONG LAB (GZ), Guangzhou, Guangdong (CN)

(72) Inventors: Ren LAI, Guangzhou, Guangdong (CN); Fuwen WEI, Guangzhou, Guangdong (CN); Aili WANG, Guangzhou, Guangdong (CN); Wenliang ZHOU, Guangzhou, Guangdong (CN)

(21) Appl. No.: 18/704,785

(22) PCT Filed: Nov. 9, 2023
(86) PCT No.: PCT/CN2023/130633

§ 371 Date: Apr. 25, 2024

(30) Foreign Application Priority Data
Dec. 2, 2022 (CN) 202211539374.1

(51) Int. Cl. A61K 8/64; A61K 8/92; A61Q 19/08; C07K 14/435

(52) U.S. Cl. CPC A61K 8/64; A61K 8/92; A61Q 19/08; C07K 14/43509; A61K 2800/91

(57) ABSTRACT

The present disclosure belongs to the technical field of cosmetic preparation and provides a modified cone snail polypeptide, preparation method and use thereof. The amino acid residues are modified on the basis of the wild-type μ-cone snail polypeptide PIIIA, and the blocking effect of μ-cone snail polypeptide on sodium channels is moderately weakened, while maintaining stability under acidic and alkaline conditions and maintaining the stability of the isoelectric point and the target specificity. The modified cone snail polypeptide provided by the present disclosure has the characteristics of high stability and low production cost. The modified cone snail polypeptide after transformation and modification retains a strong anti-wrinkle function. The modified cone snail polypeptide avoids facial muscle stiffness and paralysis resulting from excessive inhibition of skeletal muscle sodium channel Nav1.4. At the same time, the use of modified cone snail polypeptide also significantly reduces the risk of inhibiting myocardial sodium channels and brain sodium channels, and modified cone snail polypeptide is readily absorbed through the membrane.

### STATEMENT OF PRIORITY

This patent application is a national stage application of International Patent Application No. PCT/CN2023/130633, filed on Nov. 9, 2023, which claims the benefit and priority of Chinese patent application No. 202211539374.1 filed with the China National Intellectual Property Administration on Dec. 2, 2022, the disclosure of which is incorporated by reference herein in its entirety.

### STATEMENT REGARDING ELECTRONIC FILING OF A SEQUENCE LISTING

A Sequence Listing in XML format, entitled 1684-4_ST26.xml, 3,323 bytes in size, generated on Apr. 18, 2024, and filed herewith, is hereby incorporated by reference in its entirety for its disclosures.

### TECHNICAL FIELD

The present disclosure belongs to the technical field of preparation of cosmetics, and specifically relates to a modified cone snail polypeptide, preparation method, and use thereof.

### BACKGROUND

At present, Botox is widely used for skin anti-wrinkle in the medical aesthetics industry.

The anti-wrinkle mechanism of Botox is that it reduces the dynamic expression wrinkles caused by muscle stretch via blocking of the signal transmission between nerves and muscles. However, not everyone can achieve wrinkle reduction after injection of Botox. At the same time, injection of Botox may also be accompanied by side effects such as facial muscle paralysis and weakness, muscle atrophy, and swallowing difficulty. Therefore, the modification and synthesis of new peptide-based wrinkle-smoothing products represents broad application prospects.

Conotoxins are a group of biologically active polypeptides isolated and purified from the venom of the Conus genus, which are also known as conopeptides. Most conotoxins are composed of 10 to 40 amino acid residues and contain one or more disulfide bonds. They have the advantages of small molecular weight, specific target site, and strong activity. They can specifically act on voltage-gated ion channels and ligand-gated ion channels. They are important molecular probes for ion channel research and templates for the development of new drugs.

According to the pharmacological targets of conotoxins, conotoxins can be divided into pharmacological families such as α, μ, ω, κ, δ, ψ, σ, ρ, γ, vasopressin, convulsant, and sleep peptide. Among them, μ-conotoxins can specifically block voltage-gated sodium channel, thereby inhibiting the generation of action potentials. The connection mode of the disulfide bridge in the μ-conotoxins includes “C1-C4, C2-C5, and C3-C6”. Currently, more than 20 μ-conotoxins have been isolated, and they mainly act on the skeletal muscle sodium channel Nav1.4 and can also act on brain sodium channel Nav1.2, myocardial sodium channel Nav1.5, and pain-related sodium channels Nav1.7 and Nav1.8.

The significant effect of μ-conotoxin on the skeletal muscle sodium channel Nav1.4 can exert a very powerful muscle relaxation effect, that is, a rapid anti-wrinkle effect. Presently, there is a commercialized conus anti-wrinkle peptide: XEP-018. This conus anti-wrinkle peptide can be used in various anti-aging personal care products; for example, eye care products, which can visibly remove eye wrinkles and crow's feet; and facial care, which can visibly remove wrinkles on the forehead caused by muscle contraction.

Currently, the existing μ-cone snail polypeptides have the following defects when used in products such as pharmaceuticals and cosmetics:
Excessive inhibition of skeletal muscle sodium channel Nav1.4 leads to facial muscle stiffness and paralysis; and
The inhibition of skeletal muscle and myocardial sodium channels causes disturbance of normal membrane electrical signals and produces serious adverse reactions.

### SUMMARY

In view of this, an object of the present disclosure is to provide a modified cone snail polypeptide, preparation method and use thereof. The modified cone snail polypeptide of the present disclosure can avoid facial muscle stiffness and paralysis caused by excessive inhibition of skeletal muscle sodium channel Nav1.4. At the same time, the modified cone snail polypeptide also significantly reduces the risk of inhibition of myocardial sodium channels and brain sodium channels.

The present disclosure provides a modified cone snail polypeptide and the modified cone snail polypeptide has the amino acid sequence set forth in SEQ ID NO: 1.

Preferably, N-terminus of the modified cone snail polypeptide is acetylated.

Preferably, the modified cone snail polypeptide has an isoelectric point of 9.01, and the modified cone snail polypeptide has a relative molecular mass of 2,496.92 Da.

The present disclosure also provides a method for preparing the modified cone snail polypeptide described as above scheme, which includes the step of synthesizing the modified cone snail polypeptide using a solid phase synthesis method or a recombinant prokaryotic expression method.

The present disclosure also provides use the modified cone snail polypeptide or the cone snail polypeptide obtained by the preparation method described as above in the preparation of an anti-wrinkle and/or wrinkle-smoothing product.

The present disclosure also provides use of the modified cone snail polypeptide or the cone snail polypeptide obtained by the preparation method described as above in the preparation of a sodium ion channel inhibition product.

Preferably, the product is a cosmetics product and/or a pharmaceutical product.

Preferably, the sodium channel includes one or more of skeletal muscle sodium channel, cardiac muscle sodium channel, and ceramic sodium channel.

The present disclosure also provides an anti-wrinkle and/or wrinkle-smoothing cosmetic including the modified cone snail polypeptide or the modified cone snail polypeptide obtained by the method described above.

Preferably, the cosmetics further include a cosmetic auxiliary material and/or additional cosmetic raw materials, and the cosmetic auxiliary material is selected from one or more of a moisturizer, an emulsifier, mineral oil, vegetable oil, a thickener, a pH regulator, and a preservative. The additional cosmetic raw materials are selected from one or more of an additional polypeptide, a plant extract, a cytokine, and a vitamin.

### Beneficial Effects

The present disclosure provides a modified cone snail polypeptide having the amino acid sequence set forth in SEQ ID NO: 1.

The modified cone snail polypeptide of the present disclosure is obtained by modifying the amino acid residues of the wild-type μ-cone snail polypeptide (μ-conotoxin) PIIIA. Specifically, both the arginine (R) at positions 2 and 12 of the wild-type μ-cone snail polypeptide PIIIA, which interact with the skeletal muscle Nav1.4 channel, are selected and substituted for alanine (A). At the same time, in order to maintain the isoelectric point stability and target specificity, both glutamine (Q) at positions 1 and 15 are substituted for asparagine (N), leucine at position 3 is substituted for isoleucine, and lysine (K) at position 17 is substituted for arginine (R), thus affording the designed and modified cone snail polypeptide YULUO19. In the present disclosure, the amino acid residues on the wild-type μ-cone snail polypeptide PIIIA are modified, which moderately weakens the blocking effect of μ-cone snail polypeptide on sodium channels, while maintaining stability under acidic and alkaline conditions and maintaining isoelectric point stability and target specificity. The modified cone snail polypeptide provided by the disclosure has the characteristics of high stability and low production cost. The modified cone snail polypeptide after transformation and modification retains a strong anti-wrinkle function. The use of modified cone snail polypeptide enables avoidance of facial muscle stiffness and paralysis resulting from excessive inhibition of skeletal muscle sodium channel Nav1.4. At the same time, the use of modified cone snail polypeptide also significantly reduces the risk of inhibiting myocardial sodium channels and brain sodium channels, and modified cone snail polypeptide is readily absorbed through the membrane.

### BRIEF DESCRIPTION OF THE DRAWINGS

In order to describe the technical solutions in the embodiments of the present disclosure more clearly, the drawings will be briefly described below.

FIGS. 1A-1C provide diagrams of the simulated structure of the cone snail polypeptide PIIIA and the skeletal muscle sodium channel Nav1.4.

FIGS. 2A-2H show the results of functional tests for the wild-type and the modified polypeptides.

### DETAILED DESCRIPTION OF THE EMBODIMENTS

In order to make the objectives, technical solutions, and advantages of the present disclosure clearer, the technical solutions in the embodiments of the present disclosure will be described clearly and thoroughly below. In case specific conditions are not specified in the examples, the examples will be practiced according to the conventional conditions or the conditions recommended by the manufacturer. In case the manufacturers of the reagents or instruments used in the experiments are not indicated, reagents or instruments will be conventional products that can be purchased commercially.

The disclosure provides a modified cone snail polypeptide, where the modified cone snail polypeptide has the amino acid sequence set forth in SEQ ID NO: 1. Specifically, the amino acid sequence is: NAICCGFPRSCASRNCRPHRCC (SEQ ID NO:1).

The modified cone snail polypeptide provided by the disclosure is a tightly folded small peptide containing three pairs of disulfide bonds. The modified cone snail polypeptide contains an α-helix, a β-hairpin, and a compact CS αβ motif (cysteine stabilized αβ motif) composed of several turns, where Arg and Lys are key amino acids.

In the present disclosure, the N-terminus of the modified cone snail polypeptide is preferably modified by acetylation, and the primary structure of the entire amino acid sequence is:

N (Ac)-Asn-Ala-Ile-Cys-Cys-Gly-Phe-Pro-Arg-Ser-Cys-Ala-Ser-Arg-Asn-Cys-Arg-Pro-His-Arg-Cys-Cys-C(SEQ ID NO:2).

In the present disclosure, when the primary sequence of the modified cone snail polypeptide is synthesized, the N-terminus is protected through acetylation, which helps to improve the stability of the polypeptide. The modified peptide has a better lipid-water partition coefficient and is easily absorbed through the membrane.

The cone snail polypeptide are obtained by modifying the amino acid residues of the wild-type μ-cone snail polypeptide (μ-conotoxin) PIIIA, which moderately weakens the blocking effect of μ-cone snail polypeptide on sodium channels, while maintaining stability under acidic and alkaline conditions and maintaining isoelectric point stability and target specificity.

The wild type μ-cone snail polypeptide has multiple pairs of amino acid residues that can directly interact with skeletal muscle sodium channel Nav1.4. In the present disclosure, several positively charged amino acid residues of the μ-cone snail polypeptide are modified to reduce the activity of μ-cone snail polypeptide while maintaining its partial function of inhibiting the skeletal muscle Nav1.4 channel, which retains necessary nerve impulse conduction function and achieves moderate muscle relaxation effect.

Specifically, in the present disclosure both the arginine (R) at positions 2 and 12 of the wild-type μ-cone snail polypeptide PIIIA, which interact with the skeletal muscle Nav1.4 channel, are selected and substituted for alanine (A). At the same time, in order to maintain the isoelectric point stable and target-specific, both glutamine (Q) at positions 1 and 15 are substituted for asparagine (N), leucine at position 3 is substituted for isoleucine, and lysine (K) at position 17 is substituted for arginine (R), thus affording the designed and modified cone snail polypeptide YULUO19.

In some embodiments of the present disclosure, the modified cone snail polypeptide sequence has a length of 22 amino acids, an isoelectric point of 9.01, and a relative molecular mass of 2,496.92 Da, whereas wild-type PIIIA (the wild-type μ-cone snail polypeptide) has an isoelectric point of 9.49 and a molecular weight of 2,597.13 Da.

The present disclosure also provides a method for preparing the modified cone snail polypeptide described as above scheme, which includes the step: of synthesizing the modified cone snail polypeptide using a solid phase synthesis method or a recombinant prokaryotic expression method.

In the present disclosure, when a solid-phase synthesis method is adopted, it is preferred to firstly synthesize a crude modified cone snail polypeptide and then subject the crude polypeptide to refolding for higher-order structure.

In some embodiments of the present disclosure, a glutathione redox method is preferably used for refolding.

After the crude polypeptide is subjected to refolding for higher-order structure, it is preferred to further include purifying the refolded polypeptide.

In some embodiments of the present disclosure, the purifying preferably includes purifying by desalting through a high-performance liquid chromatogram (HPLC) reverse-phase column chromatography.

When the recombinant prokaryotic expression method is used to synthesize the cone snail polypeptide, it is preferred to firstly construct a prokaryotic expression plasmid, express the prokaryotic expression plasmid in Escherichia coli, and perform subsequent isolation and purification.

The present disclosure also provides use of the modified cone snail polypeptide or the cone snail polypeptide obtained by the preparation method described as above in the preparation of anti-wrinkle and/or wrinkle-smoothing products.

The present disclosure also provides use of the modified cone snail polypeptide or the cone snail polypeptide obtained by the preparation method described as above in the preparation of a sodium ion channel inhibition product.

The modified cone snail polypeptide of the present disclosure retains a strong anti-wrinkle function and the use of the modified cone snail polypeptide can avoid facial muscle stiffness and paralysis caused by excessive inhibition of the skeletal muscle sodium channel Nav1.4. At the same time, the use of the modified cone snail polypeptide also significantly reduces the risk of inhibition of myocardial sodium channels and brain sodium channels.

In some embodiments of the present disclosure, the product is preferably a cosmetic product and/or a pharmaceutical product.

In some embodiments of the present disclosure, the sodium channel is preferably selected from a skeletal muscle sodium channel, a cardiac muscle sodium channel, and a ceramic sodium channel.

In some embodiments of the present disclosure, the skeletal muscle sodium channel is preferably skeletal muscle sodium channel Nav1.4.

The present disclosure also provides an anti-wrinkle and/or wrinkle-smoothing cosmetic including the modified cone snail polypeptide or the modified cone snail polypeptide obtained by the method described above.

In some embodiments of the present disclosure, the cosmetic product preferably further includes a cosmetic auxiliary material and/or an additional cosmetic raw material.

In some embodiments of the present disclosure, the cosmetic auxiliary material is preferably selected from one or more of a moisturizer, an emulsifier, mineral oil, vegetable oil, a thickener, a pH adjuster and a preservative.

In some embodiments of the present disclosure, the moisturizer is preferably selected from one or more of glycerin, polyol, sodium hyaluronate, ceramide, trehalose, polysorbate-30 and an amino acid moisturizing agent.

In some embodiments of the present disclosure, glycerin acts as both a moisturizer and an antioxidant.

In some embodiments of the present disclosure, the emulsifier is preferably selected from one or more of lanolin, polyglyceryl-10 stearate, octylmethicone, polydimethylsiloxane, polymethylsilsesquioxane, babassu seed oil and phytosteryl oleate.

In some embodiments of the present disclosure, the thickener is preferably selected from one or more of carbomer, hydroxyethyl cellulose, xanthan gum, and hydroxyethyl acrylate/sodium acryloyldimethyltaurate copolymer.

In some embodiments of the present disclosure, the pH adjuster is preferably selected from one or more of citric acid, sodium citrate, lactic acid, sodium lactate, triethanolamine, and arginine.

In some embodiments of the present disclosure, the preservative is preferably selected from one or more of 1,2-hexanediol, p-hydroxyacetoenone (SymSave® H) and ethylhexylglycerin.

In some embodiments of the present disclosure, the additional cosmetic raw material preferably includes one or more of an additional polypeptide substance, a plant extract, a cytokine, and a vitamin.

In some embodiments of the present disclosure, the additional polypeptide substance is preferably selected from one or more of camosine, pentapeptide-3, glutathione, anserine, and ophidine.

In some embodiments of the present disclosure, the plant extract preferably includes oat kernel extract and/or Dendrobium officinale extract.

In the present disclosure, there is no special limitation on the methods of extracting oat kernel extract and Dendrobium officinale extract.

In some embodiments of the present disclosure, the vitamin is preferably selected from one or more of vitamin B3, vitamin C and vitamin E.

In The present disclosure, there is no special limitation on the proportion of the cosmetic auxiliary material and the additional cosmetic raw material, and adaptive combination can be carried out as needed.

The technical solutions in the present disclosure will be clearly and completely described below with reference to the examples of the present disclosure.

The purpose of the present disclosure is to provide a polypeptide product for skin anti-wrinkle that is safe and reliable, has definite curative effect, has good stability and is easy to produce through structural modification and design.

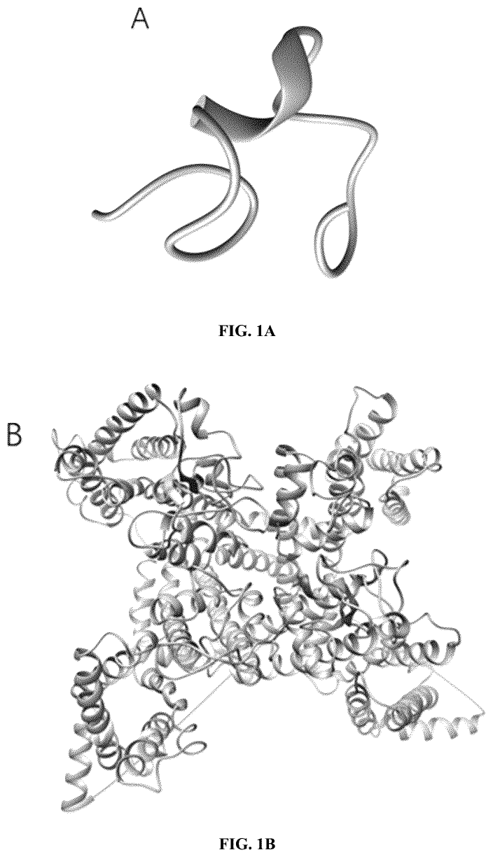

Based on the cone snail polypeptide (μ-conotoxin PIIIA) with strong side effects, the structure simulation software Rosetta was used to simulate the 3D structure of PIIIA (FIG. TA) and the skeletal muscle sodium channel Nav1.4 (FIG. 1B and FIG. 1C). By analyzing the high-affinity complex model, direct interactions between multiple pairs of PIIIA polypeptides and key amino acid residues in the Nav1.4 channel were obtained. The amino acid residues that directly interact are shown in Table 1 below.

TABLE 1
List of key amino acid residues for direct interaction

| PIIIA | R2 | R12 | R14 | K17 | R20 |
|---|---|---|---|---|---|
| Nav1.4 | W761 | E758 | E755 | D1241 | D1541 |

From the analysis of the information in the above table, it is concluded that:

(1) There may be multiple pairs of interactions between the cone snail polypeptide μ-conotoxin PIIIA and the skeletal muscle sodium channel Nav1.4. The direct interaction of these amino acids serves the basis for the high affinity of PIIIA in the sodium channel.

(2) By modifying the positively charged amino acid residues on PIIIA, the blocking effect of PIIIA on sodium channels can be moderately weakened, while maintaining stability under acidic and alkaline conditions.

(3) On wild-type PIIIA, arginine (R) at positions 2 and 12, which interact with the skeletal muscle channel Nav1.4, were selected and simultaneously modified into glycine (G). At the same time, in order to maintain the stability of the isoelectric point and target specificity, Glutamine (Q) at positions 1 and 15 were modified into asparagine (N), leucine at position 3 was modified into isoleucine, and lysine (K) at position 17 was modified into arginine (R), and then the designed modified cone snail polypeptide was obtained, which was designated as YULUO19.

### Experimental Example 1

This example provides a modified cone snail polypeptide (YULUO19), and the primary structure of its full amino acid sequence was:

N(Ac)-Asn-Ala-Ile-Cys-Cys-Gly-Phe-Pro-Arg-Ser-Cys-Ala-Ser-Arg-Asn-Cys-Arg-Pro-His-Arg-Cys-Cys-C(SEQ ID NO:2). This modified cone snail polypeptide was a tightly folded small peptide containing three pairs of disulfide bonds.

The preparation method of the modified cone snail polypeptide was as follows:

(1) synthesizing a crude polypeptide using a solid-phase synthesis method according to the above-mentioned amino acid sequence as designed; and

(2) subjecting the linear polypeptide to refolding for higher-order structure. The synthesized linear polypeptide was refolded using a glutathione redox method, which specifically included the following steps: dissolving 10 mg of the linear polypeptide synthesized in step (1) in 100 ml solution (pH=8.0) containing 5 mM GSH and 0.5 mM GSSG and 0.1 M Tris HCl and 0.1 M NaCl, placing the resulting mixture in an incubator at 25° C. for 24 hours to obtain a refolded protein solution. The refolding effect was detected by RP-HPLC and the eluates corresponding to the elution peaks were collected. The purity and refolding results were detected by mass spectrometry.

(3) The refolded protein solution obtained in step (2) was purified by desalting through an HPLC reverse-phase column chromatography, and the purity of the purified refolded protein solution was identified until the purity of the polypeptide was not less than 95%.

Method for HPLC Purification and Identification: 10 ml of refolded protein solution was filtered through a 0.22-μm membrane, mobile phase A was 0.10% trifluoroacetic acid-water, mobile phase B was 0.1% trifluoroacetic acid-acetonitrile, the sample loading was started after the baseline was stable, the column was alkyl bonded phase C18 column (4.6 mm×300 mm, the gel size was 5 μm, and the pore size was 100 Å), and the binary mobile phase gradient elution system was used for linear gradient elution, that is, the content of mobile phase B in the eluent increased linearly from 0% to 100% within 200 min, with a flow rate of 1 mL/min and a detection wavelength of 280 nm, measurement temperature of 25° C.

(4) The collected single peaks were determined by matrix-assisted laser desorption ionization time-of-flight (MALDI-TOF) mass spectrometry, with a molecular weight of 2490 Da after refolding.

The determination method was as follows: the purified polypeptide obtained in step (4) was dissolved in deionized water, and prepared into a 1 μM solution, 10 μL was taken and mixed with the equal volume of saturated matrix solution (α-cyano-4-hydroxycinnamic acid was dissolved in a 50% acetonitrile solution containing 0.1% trifluoroacetic acid to prepare a saturated solution, the resulting saturated solution was centrifuged, and the supernatant was collected), and determination was followed.

(5) The isoelectric point of the purified polypeptide was determined by isoelectric focused electrophoresis, which was 9.01, and the amino acid sequence structure of the purified polypeptide was determined by an automatic amino acid sequencer, which was determined as NAICCGFPRSCASRNCRPHRCC (SEQ ID NO:1).

### Experimental Example 2

In this experimental example, the dispersion of the polypeptide YULUO19 prepared in Example 1 in a lipid substance was tested.

The determination of homogeneity and dispersion of peptide YULUO19 in lanolin was taken as an example. One thousand grams of lanolin and 1000 mg of polypeptide YULUO19 were mixed homogenously at room temperature in an SHW/R mobile high-shear emulsifier, with a stirring speed of 120 rpm/min and a stirring time of 30 min. After the mixing, the mixture was dispensed and allocated into 5 ml for each tube. The theoretical content of peptide YULUO19 under this condition was 1 mg/g. 20 allocations were taken, and an appropriate amount of 1 g mixture (equivalent to about an amount of 0.1 mg YULUO19 peptide, 10 ml) was placed in a flask, then 20% ethanol solution was added for dissolution (sonication was conducted if necessary) and diluted to volume. Two millimeter of diluted solution was pipetted and was quantification for protein content using a Folin phenol method.

The experimental results showed that the average content of the polypeptide YULUO19 in 20 samples was 0.90±0.05 mg/g, and the content range of each sample fell within 90% of the theoretical content.

It was showed that the polypeptide YULUO19 designed by the present disclosure had good dispersion in the lipid environment. That is, the polypeptide YULUO19 can be used in the development of anti-wrinkle skincare products.

### Experimental Example 3

In this experimental example, the electrophysiological function of the peptide prepared in Example 1 YULUO19 was tested.

The recording of the voltage-gated sodium channel was under extracellular high-sodium system with extracellular fluid (mM): 140 mM NaCl, 3 mM KCl, 1 mM MgCl2, 1 mM CaCl₂), 10 mM HEPES (pH was adjusted to 7.3 with NaOH); Intracellular fluid (mM): 140 mM CsF, 1 mM EGTA, 10 mM NaCl, 3 mM KCl, 10 mM MgCl2 (pH was adjusted to 7.3 with CsOH).

The recording of the sodium channel was as follows: clamping voltage: 80 mV for 20 ms; test voltage: 10 mV for 50 ms; and clamping voltage: 80 mV for 20 ms. This recording was repeated in a continuous cycle form until the current became stable.

Cells containing transiently expressed Nav1.4 were placed in front of the 8-tube strip of the RSC-200 (BioLogic) Rapid Perfusion Dosing System, and the Bath Solution channel was turned on. By switching different channels, 100 nM wild-type PIIIA and 100 nM polypeptide YULUO19 were injected.

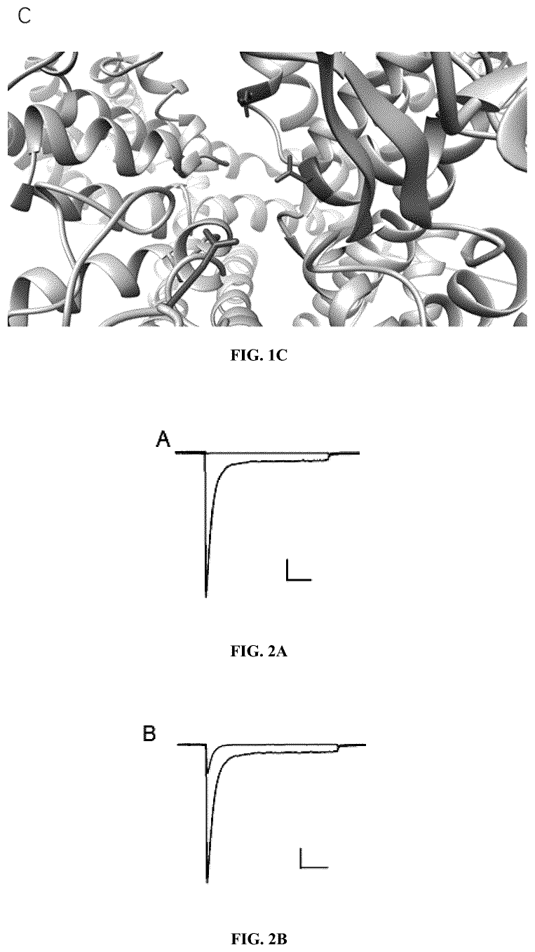

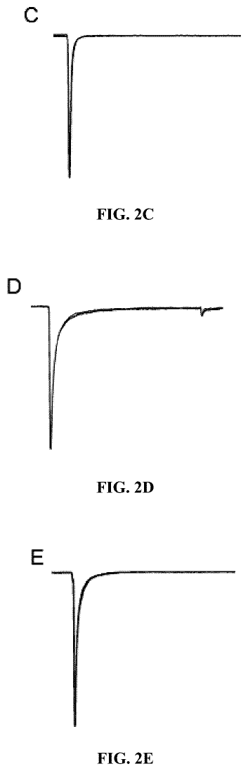

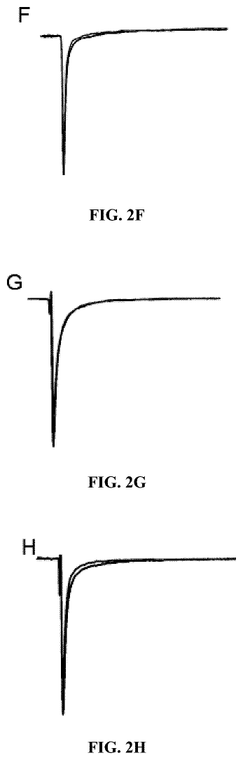

The results are shown in FIGS. 2A-2H and it can be seen that wild-type PIIIA completely inhibited the current in Nav1.4 (FIG. 2A). The polypeptide YULUO19 partially inhibited the current in Nav1.4 (FIG. 2B) by about 90%, which retained about 10% of electrical conduction in the muscle. At the same time, the polypeptide YULUO19 at this concentration was virtually inactive to other sodium channels that assume important physiological functions, such as Nav1.2 (FIG. 2C), Nav1.5 (FIG. 2D), Nav1.7 (FIG. 2E) (FIGS. 2C-2E), while wild-type PIIIA showed inhibition effect in channels such as Nav1.2 (FIG. 2F), Nav1.5 (FIG. 2G), and Nav1.7 (FIG. 2H) (FIGS. 2F-2H). Thus, the modified peptide ensured target specificity.

From the above results, it can be seen that the inventor has allowed the polypeptide to moderately inhibit muscle contraction through creative design and modification, and the polypeptide will not act on other ion channels such as Nav1.2, Nav1.5 and Nav1.7 and cause serious side effects.

### Experimental Example 4

In this experimental example, the polypeptide YULUO19 prepared in Example 1 was tested for cytotoxicity.

In this experimental example, the MTT method was used to detect the toxicity of polypeptide YULUO19 on the human skin fibroblasts HFF-1.

The human skin fibroblasts HFF-1 were purchased from Kunming Cell Bank. The fibroblasts were cultured in DMEM containing 15% fetal bovine serum and bispecific antibodies (100 U/ml each for penicillin and streptomycin), and digested with 0.25% trypsin after the cells fully grew. The fibroblasts were washed twice with the medium described above, and the cells were resuspended. After cell counting, 100 μl of cell suspension was added to a 96-well cell culture plate to bring the number of cells per well to 105. The polypeptide YULUO19 prepared in Example 1 was added, the same volume of sterilized ultrapure water was added to the control group, the temperature was set at 37° C., and culture was conducted in an incubator purged with 5% CO₂ for 24h. At the end of the culture, 20 μl of 5 mg/ml MTT solution (prepared using cell culture PBS buffer) was added to each well of the 96-well cell culture plate, the culture was continued for 5 h. The liquid in the wells was aspirated with a syringe, and 100 μl DMSO was added to each well and pipetted several times with a pipette to completely dissolve the purple crystals. Light absorption was then detected using a microplate reader at a wavelength of 490 nm and a reference wavelength of 630 nm.

TABLE 2
Statistical table of toxicity of polypeptide
YULUO19 to HFF-1 cells

| Polypeptide YULUO19 | |
|---|---|
| concentration (g/ml) | Cytotoxicity % |
| 1 | 0.00 |
| 50 | 1.25 ± 0.32 |
| 200 | 2.67 ± 0.48 |

The results are shown in Table 2 and indicate that the cytotoxicity of the polypeptide YULUO19 at a concentration of 200 μg/ml was only 2.67%, indicating that the polypeptide YULUO19 had very little cytotoxicity to human skin fibroblasts and will not harm normal skin cells in the human body. Thus, it is very conducive to its further development and application.

### Experimental Example 5

In this experimental example, the anti-wrinkle function of the polypeptide YULUO19 prepared in Example 1 was tested.

In this experiment, the effect of skin polypeptide YULUO19 on wrinkles caused by UVB irradiation was determined. The UVB energy irradiated to the dorsal side of each mouse was controlled by varying the UV irradiation time. The minimum erythema dose (MED) per mouse was approximately 36 mJ/cm². Polypeptide YULUO19 (10 ng and 100 ng/mouse) was applied topically to the back of each mouse daily for 12 weeks. The initial dose of UVB was set at 36 mJ/cm², increased to 54 mJ/cm² at weeks 1-4, increased to 72 mJ/cm² at weeks 4-7, increased to 108 mJ/cm² at weeks 7-10, and finally increased to 122 mJ/cm² at weeks 10-12. The frequency of UVB irradiation was set to three times a week, followed by topical application of excipients (blank control and UV control) and polypeptide YULUO19.

The grading criteria were as follows: 0—no rough wrinkles; 2—some shallow, rough wrinkles were observed in the skin area of the back (Bisset Level 1); 4—shallow, rough wrinkles were observed on the entire back skin (Bisset Level 2); and 6—some deep and long wrinkles were observed on the skin of the back (Bisset Level 3).

TABLE 3
Scoring of anti-wrinkle effect by
anti-wrinkle polypeptide YULUO19

| | Group | | | |
|---|---|---|---|---|
| | | | Polypeptide | Polypeptide |
| | Blank | UV | YULUO19 | YULUO19 |
| | control | control | (10 ng) | (100 ng) |
| Wrinkle | 0.0 ± 0.0 | 3.96 ± 0.63 | 1.16 ± 0.22** | 2.42 ± 0.38** |
| score at | | | | |
| week 6 | | | | |
| Wrinkle | 0.0 ± 0.0 | 4.94 ± 0.74 | 1.47 ± 0.35** | 2.68 ± 0.52** |
| score at | | | | |
| week 9 | | | | |

**P < 0.05

Table 3 shows that the polypeptide YULUO19 designed in Example 1 has good anti-wrinkle effect compared to the control group.

Although the above embodiments have described the present disclosure in detail, it is merely a part but not all of the embodiments of the present disclosure, and one can obtain other embodiments without inventive efforts based on the embodiments disclosed herein. The obtained embodiments shall fall within the scope of protection of the present disclosure.

1. A modified cone snail polypeptide, wherein the modified cone snail polypeptide has the amino acid sequence set forth in SEQ ID NO: 1.

2. The modified cone snail polypeptide according to claim 1, wherein N-terminus of the modified cone snail polypeptide is modified by acetylation.

3. The modified cone snail polypeptide according to claim 1, wherein the modified cone snail polypeptide has an isoelectric point of 9.01 and a relative molecular mass of 2,496.92 Da.

4. A method for preparing the modified cone snail polypeptide according to claim 1, comprising a step of synthesizing the modified cone snail polypeptide using a solid synthesis method or a recombinant prokaryotic expression method.

5. The method according to claim 4, wherein the solid phase synthesis method comprises: synthesizing a crude cone snail polypeptide, and then subjecting the crude cone snail polypeptide to refolding for a higher-order structure to obtain the cone snail polypeptide.

6. The method according to claim 5, wherein a method for the refolding comprises refolding by a glutathione redox method.

7. The method according to claim 4, further comprising purifying a refolded polypeptide after performing the refolding for higher-order structure.

8. The method according to claim 7, wherein the purifying comprises purification by desalting through high performance liquid chromatogram (HPLC) reverse-phase column chromatography.

9. The method according to claim 4, wherein the recombinant prokaryotic expression method comprises: constructing a prokaryotic expression plasmid, expressing the prokaryotic expression plasmid in Escherichia coli, and then performing subsequent isolation and purification; and wherein
the prokaryotic expression plasmid comprises a nucleotide sequence encoding the cone snail polypeptide.

10. A method for preparing an anti-wrinkle and/or wrinkle-smoothing product, comprising adding the modified cone snail polypeptide of claim 1 to the anti-wrinkle and/or the wrinkle-smoothing product.

11. A method for preparing a sodium ion channel inhibition product, comprising adding the modified cone snail polypeptide of claim 1 to the sodium ion channel inhibition product.

12. The method according to claim 10, wherein the product is a cosmetic product and/or a pharmaceutical product.

13. The method according to claim 11, wherein the sodium channel is selected from one or more of a skeletal muscle sodium channel, a cardiac muscle sodium channel, and a ceramic sodium channel.

14. An anti-wrinkle and/or wrinkle-smoothing cosmetic product, comprising the modified cone snail polypeptide according to claim 1.

15. The cosmetic product according to claim 14, wherein the cosmetic product further comprises a cosmetic auxiliary material and/or an additional cosmetic raw material; the cosmetic auxiliary material is selected from one or more of a moisturizer, an emulsifier, a mineral oil, a vegetable oil, a thickener, a pH adjuster and a preservative; the additional cosmetic raw material and the additional cosmetic raw material is selected from one or more of an additional polypeptide substance, a plant extract, a cytokine, and a vitamin.

16. The cosmetic product according to claim 15, wherein the moisturizer is preferably selected from one or more of glycerin, polyol, sodium hyaluronate, ceramide, trehalose, polysorbate-30 and an amino acid moisturizing agent;
the emulsifier is selected from one or more of lanolin, polyglyceryl-10 stearate, octylmethicone, polydimethylsiloxane, polymethylsilsesquioxane, babassu seed oil and phytosteryl oleate;
the thickener is selected from one or more of carbomer, hydroxyethyl cellulose, xanthan gum, and hydroxyethyl acrylate/sodium acryloyldimethyltaurate copolymer;
the pH adjuster is selected from one or more of citric acid, sodium citrate, lactic acid, sodium lactate, triethanolamine, and arginine;
the preservative is selected from one or more of 1,2-hexanediol, p-hydroxyacetoenone (SymSave® H) and ethylhexylglycerin;
the additional polypeptide substance is selected from one or more of carnosine, pentapeptide-3, glutathione, anserine, and ophidine;
the plant extract includes oat kernel extract and/or Dendrobium officinale extract;
and the vitamin is selected from one or more of vitamin B3, vitamin C and vitamin E.

17. The method according to claim 11, wherein the product is a cosmetic product and/or a pharmaceutical product.

18. An anti-wrinkle and/or wrinkle-smoothing cosmetic product, comprising the modified cone snail polypeptide according to claim 2.

19. An anti-wrinkle and/or wrinkle-smoothing cosmetic product, comprising the modified cone snail polypeptide according to claim 3.
